(19) United States
(12) Patent Application Publication
(10) Pub. No.: US 2012/0108944 A1
(43) Pub. Date: May 3, 2012

(54) In Vivo 1H Magnetic Resonance Spectroscopy For The Diagnosis Of Testicular Function And Disease

(75) Inventors: Paul Turek, San Francisco, CA (US); John Kurhanewicz, South San Francisco, CA (US)

(73) Assignee: The Regeents of the University of California, Oakland, CA (US)

(21) Appl. No.: 13/265,815

(22) PCT Filed: Apr. 22, 2010
(86) PCT No.: PCT/US10/32119

§ 371 Date: Jan. 9, 2012

Related U.S. Application Data
(60) Provisional application No. 61/171,758, filed on Apr. 22, 2009.

(51) Int. Cl. A61B 5/055

(52) U.S. Cl. 600/410

(57) ABSTRACT

This invention relates to the use of ¹H magnetic resonance spectroscopy for the diagnosis of testicular function and disease.

### RELATED APPLICATIONS

This application is related to and claims the benefit of Provisional Patent Application Ser. No. 61/171,758 filed on 22 Apr. 2009.

### FIELD

This invention relates generally to the field of analytical tools and techniques in medical research and diagnosis. Specifically, it relates to the use of magnetic resonance spectroscopy to obtain a metabolic (i.e. metabolomic) profile of a testis for use in evaluating the function of the testis, in particular its ability to produce healthy sperm.

### BACKGROUND

It is estimated that 10-15% of couples are infertile and of those the cause of nearly half is male infertility. In the population of infertile males 6-10% have been found to produce no ejaculated sperm due to testicular failure, termed non-obstructive azoospermia (NOA). Some NOA men have small numbers of sperm in the testicle, generally located in “pockets” or focal areas within the testis, which can be extracted using sperm retrieval techniques. Retrieved sperm is then used with in vitro fertilization and intracytoplasmic sperm injection for biological pregnancies.

Unfortunately, determining which NOA men have retrievable sperm in the testicle is a major clinical challenge. Potential predictors of successful sperm retrieval that have been investigated include infertility diagnosis, history of ejaculated sperm, serum hormones, testis volume and testis biopsy histology. Although all are poor predictors, the testicular biopsy best predicts the presence or absence of sperm. Biopsy, however, is an invasive procedure that is not only unattractive from the patient's perspective but that invites the potential complications attendant to any invasive procedure while offering information only about the biopsied area and not about usable pockets of sperm that may exist elsewhere in the testis. In view of these and other factors, reproductive urologists have developed alternative sperm sampling strategies, such as diagnostic fine needle aspiration testis mapping and multi-biopsy microdissection sperm extraction. Even with these advanced techniques, some of which are still quite invasive, sperm is found in only 60-65% of NOA men.

What is needed is a non-invasive technique for definitively identifying viable sperm in NOA men. The current invention provides such a technique. While the foregoing is the presently preferred use of this invention, it is anticipated that the techniques and analysis disclosed herein will be equally applicable to the diagnosis of numerous other conditions relating to the function of the testes both in health and disease.

### SUMMARY

Thus, in one aspect the current invention relates to a method comprising obtaining an in vivo ¹H magnetic resonance spectroscopy spectrum of a testis of a subject; comparing the subject testis spectrum with a spectrum from that of a control subject; and, diagnosing the condition of the testis of the subject.

In an aspect of this invention, the condition of the testis being diagnosed comprises the presence of sperm and attendant fertility potential.

In an aspect of this invention, diagnosing the presence of sperm comprises comparing phosphocholine, taurine and other potentially relevant metabolic signals of the two spectra.

In an aspect of this invention, the subject has undergone a surgical intervention for treatment of infertility

In an aspect of this invention, the surgical intervention is a varicocele repair, vasectomy, orchidopexy, vasectomy reversal or other related scrotal surgery.

In an aspect of this invention, the subject has undergone medical treatment for infertility.

In an aspect of this invention, the medical treatment comprises administration of clomiphene citrate, tamoxifen or other selective estrogen response modulators, and other hormonal (e.g. FSH, LH injections, dopamine antagonists, steroids) or non-hormonal (e.g. vitamin supplements, herbal remedies) manipulation of sperm production.

In an aspect of this invention, obtaining the subject spectrum and the control subject spectrum comprises employing a spectrum-enhancing technique.

In an aspect of this invention, the spectrum-enhancing technique is selected from the group consisting of water suppression, lipid suppression, stimulated echo acquisition mode (STEAM) spectroscopy, point resolved spectroscopy (PRESS), chemical shift imaging (CSI), magnetic resonance spectroscopic imaging (MRSI), short T1 inversion recovery (STIR), high resolution MRS, magic angle spinning and any combination thereof.

In an aspect of this invention, the condition of the testis being diagnosed is the effect of exposure to a pharmacological agent, exposure to a toxic agent, exposure to abnormal physical conditions, exposure to radiation therapy, exposure to chemotherapy, exposure to a disease or any combination of the foregoing on the production of sperm.

In an aspect of this invention, the condition of the testis being diagnosed is the presence of testicular disease or systemic disease or exposures that impact on testis function, and in particular, sperm production.

In an aspect of this invention, the testicular disease is cancer, cryptorchidism, orchitis, effect of varicocele, medications or other exposures such as prolonged blockage from vasectomy or other excurrent duct obstruction, and systemic toxins.

In an aspect of this invention, the subject and control subject are human beings.

### DETAILED DESCRIPTION

### Brief Description of the Figures

FIG. 1 comprises ¹H high resolution MRS spectra from pooled ex vivo testis biopsies from 5 men with normal spermatogenesis (top spectrum) and 5 men with absent spermatogenesis. It is readily apparent that choline-containing compounds differ dramatically between the groups indicating that these compounds are relevant biomarkers for fertility-related disease states.

FIG. 2 is a graph showing the phosphocholine (PC) concentrations (mmol/kg) in men with normal spermatogenesis, arrested sperm production (maturation arrest) and no spermatogenesis (azoospermia) due to Sertoli cell-only syndrome.

FIG. 3 is a ¹H NMR of (A) normal, (B) maturation arrested and (C) Sertoli cell-only testis biopsies from three different patients.

FIG. 4 is a two-dimensional spectral resolution of the C peak for (A) normal, (B) maturation arrested and (C) Sertoli cell-only testis biopsies from the three patients of FIG. 3.

### DISCUSSION

It is understood that use of the singular throughout this application including the claims includes the plural and vice versa unless expressly stated otherwise. That is, “a” and “the” are to be construed as referring to one or more of whatever the word modifies. Non-limiting examples are: “a testis,” which is understood to include one testis or both testes, unless it is expressly stated or is unambiguously obvious from the context that such is not intended. Likewise, without limitation, “a spectrum” may refer to a single pass spectrum, a multiple pass spectrum or an amalgamation of two or more separate spectra, again, unless it is expressly stated or absolutely obvious from the context that such is not intended. The converse is also to be taken as true, i.e., reference to an item in the plural includes the singular unless it is otherwise unambiguously clear from the context that only the plural is intended.

As used herein, all technical terminology is intended to have the meaning that would be afforded it by those skilled in the relevant art unless it is expressly stated or obvious from the words or context that a different meaning is intended. Any perceived errors, discrepancies and/or omissions in such descriptions provided herein are unintentional.

As used herein, words of approximation such as, without limitation, “about” “substantially,” “essentially” and “approximately” mean that the word or phrase modified by the term need not be exactly that which is written but may vary from that written description to some extent. The extent to which the description may vary will depend on how great a change can be instituted and have one of ordinary skill in the art still recognize the modified version as still having the properties, characteristics and capabilities of the modified word or phrase. In general, but with the preceding discussion in mind, a numerical value herein that is modified by a word of approximation may vary from the stated value by ±15%.

As used herein, the use of “preferred,” “preferably,” or “more preferred,” and the like refers to preferences as they existed at the time of filing of the patent application.

As used herein in vivo refers to operations carried out on a living organism in its natural state. That is, an in vivo magnetic resonance spectroscopy spectrum of a testis is conducted on an intact testis in its natural environment where the testis has not been subjected to any manner of invasive procedure. To the contrary, ex vivo or in vitro, the terms being considered essentially interchangeable, is used to express operations on excised, isolated tissues of a living organism.

As used herein magnetic resonance spectroscopy (MRS) refers to the same analytical technique commonly known in organic chemistry as nuclear magnetic resonance or NMR spectroscopy, in fact the terms are used interchangeable in the art and in this description. In simplest terms, MRS and NMR refer to the detection of the effect on an atomic nucleus having a characteristic spin when it is exposed to a constant magnetic field of varying radiofrequencies or vice versa, that is, the effect of varying magnetic intensity on an atomic nucleus exposed to a constant frequency. By convention, the former is the most commonly employed technique. While all nuclei with an odd number of protons, an odd number of neutrons or both will exhibit an intrinsic spin, the nuclei of most interest to organic chemists and medical researchers are ¹H, ¹³C, ¹⁹F and ³¹P. Presently preferred for the purposes of this invention is NMR probing of the ¹H nucleus.

The intensity of the magnetic field used in NMR in terms of the international unit of magnetic flux, the Tesla (T), is from about 1 to about 20 T, although use of fields up to 45 T have been reported. One Tesla is equal to about 10,000 gauss. By way of contrast, the earth's magnetic flux is about 10⁻⁴ T at ground level. For the purposes of this invention, wherein living organisms are exposed directly to a magnetic field, a field intensity of about 1 T to about 7 T is presently preferred.

As used herein, a spectrum is a readout of the response in terms of signal intensity of the target nuclei, preferably at present ¹H nuclei, as a function of frequency in the vicinity of the main resonance frequency of the nuclei at a given magnetic field strength. For example, at 21 T, ¹H resonates at 900 MHz. The frequency sweep, then would be in the vicinity of 900 MHz, so close in fact that the full range of the frequency sweep is stated in parts per million or ppm. At 3 T, a common field strength for in vivo studies, ¹H resonates at about 100 mHz. The spectrum may be the result of a single pass through the appropriate frequency range, the sum of multiple passes through that range or it may be the result of many short pulses at or near the main resonance frequency, which is referred to generally as Fourier Transform NMR.

As used herein, a “spectrum-enhancing” technique refers to any technique, be it instrumental, procedural, calculational or otherwise, that renders the most relevant portion, in view of the information being sought, of an MRS spectrum obtained from a subject more readily and more unambiguously interpretable. Currently available techniques such as, without limitation, water suppression, lipid suppression, simulated echo acquisition mode, short T1 (longitudinal or spin-lattice relaxation time) inversion recovery, high resolution MRS, high resolution magic angle spinning (HRMAS) and any combination thereof may be used to enhance the spectrum obtained for the purposes of this invention. Of course, spectrum-enhancing techniques that may arise in the future are also within the scope of this invention insofar as they are applicable to the types of information being sought by the present invention.

As used herein, a “subject” refers to living organism characterized by a sexual reproductive system that requires the presence of spermatozoa that are created in the testes of the male of the species. In particular, a subject herein is a mammal and most particularly at present the subject is human being. As used herein, a “control subject” refers to a mammal of the same species as the subject where the control subject either has healthy testes that function normally with regard to sperm production or that has impeded testis function as the result of known insult such as exposure to pharmacological agents, toxicological agents, chemotherapy, radiation therapy, abnormal physical conditions such as chronic systemic disease, excessive heat, varicocele, blockage, etc. The control may also be afflicted with a testicular or other disease that affects the function of the testes.

As used herein, diagnosis refers to the determination of the function of an organ though analysis of its MRS spectrum, along with consideration of other factors such as exposure to pharmacological or toxicological agents, excessive physical forces such heat, varicocele, blockage etc. and any other factor that the skilled diagnostician would take into consideration. Preferable at present, the organ is the testis, and the function being diagnosed is its ability to produce healthy sperm. Presently preferred is diagnosis based primarily on an in vivo ¹H MRS spectrum of the testis of the subject.

As used herein, “azoospermia,” or “azoospermic” refers to the condition where the semen contains no sperm. The term includes obstructive azoospermia, where sperm are created but cannot be mixed with the rest of the ejaculatory fluid due to a physical blockage or it may be non-obstructive azoospermia where there is a problem with spermatogenesis. In addition, severe oligospermia refers to the condition where semen contains few or unusable sperm. In these cases, testicular sperm is considered an alternative for pregnancy.

As used herein, “metabolomics” refers to the study of the unique chemical fingerprint that specific cellular processes produce as evidenced by the small molecule metabolites—generally less than 1 kDA in size—found in the cells. While a complete metabolomic profile refers to a complete characterization of the metabolic composition of a given cell at a given time, which would require the integration of a number of analytical tools and techniques, for the purposes of this invention, metabolomic profile refers to that which can be obtained from MRS spectra of the target cells.

Fully relaxed ³¹P MRS spectra of normal, ischemic and hormonally treated dogs have been shown to exhibit similar spectral patterns including peaks for phosphomonoesters (PM), phosphodiesters (PD), inorganic phosphorus (Pi) and three peaks for adenosine triphosphospahte (ATP). Ischemic testes, however, exhibited a rapid characteristic depletion of ATP and PMs and an increase in Pi compared to normal controls. This relationship has been subsequently confirmed in the rat model.

In humans, ³¹P MRS of the testes of azoospermic subjects and fertile control subjects have been compared using semen analysis and testis biopsy findings from those two groups. Peaks similar to those found in the above animal studies were observed. Additionally, the peak area ratios of PM/β-ATP, PM/PD and Pi/PM differed significantly between the fertile controls and men with no sperm count. Further, the PM/β-ATP ratio decreased significantly when men with no sperm count due to obstruction were compared to azoospermia due to testis failure. These studies clearly suggest that PM, specifically PC (phosphocholine), are important markers for metabolic profiling of the testis and that such should be able to differentiate among infertile populations of men.

While not wishing to be held to any particular theory, the basis for the above observation may be that PMs form a metabolic pool that provides for, and accurately reflects activity in, cell membrane phospholipid synthesis. This activity is believed to be linearly correlated to the rate of cell proliferation. In the testis, changes in the rate of cellular proliferation, as measured by phospholipid synthesis, are related to the number of proliferating germ cells, specifically, spermatogonia and spermatocytes undergoing meiosis to eventually become sperm. The support cells of the testis, in particular Leydig and Sertioli cells, reproduce only slowly if at all and would not be expected to contribute meaningfully to phospholipid synthesis. A healthy testis producing spermatozoa requires constant membrane synthesis and therefore harbors a large substrate pool of PMs. In azoospermic individuals with testis failure in which sperm production is poor or absent, membrane production is decreased and a smaller pool of PMs should be present. This is confirmed by the experimental results discussed above, in particular the lower PM/β-ATP ratio of azoospermic men compared to normal, fertile men.

While ³¹P MRS may be able to provide sufficient data to make the above determinations, current advances in ¹H MRS have rendered this technique of particular interest in the definition of characteristic metabolic “fingerprints,” currently referred to as metabolomic profiling, discussed above, of normal and abnormal states of human spermatogenesis. These fingerprints or profiles should be useful as biomarkers that can define clinical states of testis health and testis disease. That is, the metabolic activity resulting from germ cells (spermatogenesis) in which about 1000 sperm/sec are produced, is markedly higher than that of a somatic cells in the testis (Sertoli, Leydig and myoid cells) and ¹H MRS should be uniquely capable of detecting this difference.

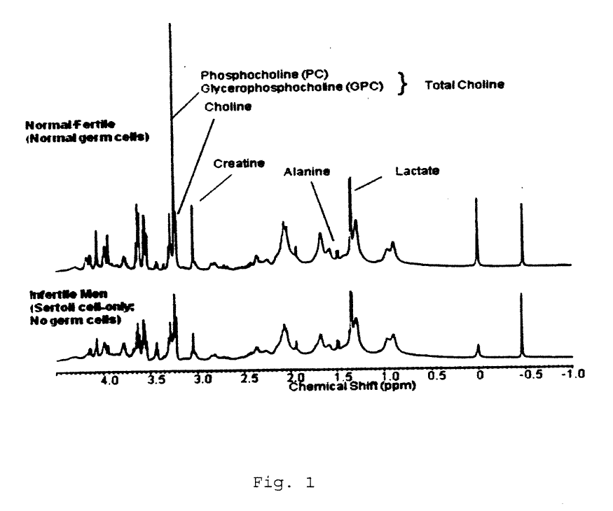

In fact, several unique metabolic biomarkers in infertile and fertile testes have been identified using ex vivo ¹H high resolution magic angle spinning MRS (HRMAS-MRS). That is, in an ex vivo analysis of testis biopsies from fertile and infertile men, the ratio of predicted critical biomarkers were compared. FIG. 1 shows the results of ¹H HRMAS-MRS on two pools of testis biopsies, one pool obtained from men exhibiting normal spermatogenesis (top spectrum) and one pool from men exhibiting an absence of spermatogenesis (bottom spectrum). As can readily be seen, choline containing compounds differ dramatically between the two pools suggesting that there are relevant biomarkers to differentiate between healthy testes and those incapable of, or capable only of reduced, sperm production. It is also noted that the spectrum, which was obtained using a 11.7 T magnet, actually reveals 19 known metabolites. While the above results render it manifest that total choline peak between the fertile and infertile men is a relevant metabolic biomarker for sperm production, differences in some or all of these other metabolites, including taurine, could provide additional biomarkers relating to the effect of exposure to various conditions such as pharmacological agents, toxicological agents, abnormal environmental or physical conditions and the like on the overall function of the testes. In addition, such biomarkers might be found indicative of a variety of testicular diseases such as cancer, cryptorchidism, orchitis, benign tumors, obstruction and the like.

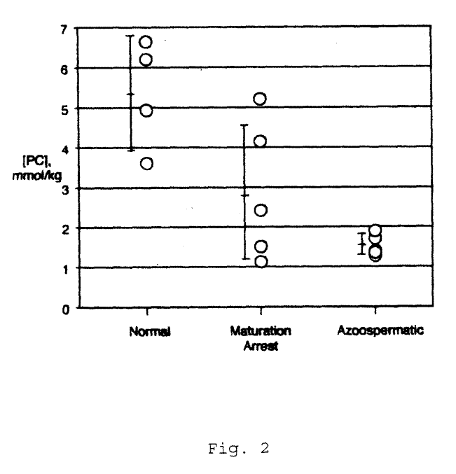

In another study, men with defective and no sperm production were compared men with normal sperm production. The phosphocholine (PC) concentration in normal testes was significantly higher than that found in testes with no sperm production, 5.35±1.37 mmol/kg versus 1.52±0.27 mmol/kg. This is shown graphically in FIG. 2. It is noted that there was no overlap in PC concentration between the two groups. This suggests the use of ¹H MRS to determine whether or not any sperm at all are being produced in the testis, an important issue in many male infertility cases in which azoospermic or severe oligospermia is present and invasive sperm retrieval techniques are being considered to find sperm for pregnancies.

Using current clinical MR scanners (1.5-3.0 T), it should also be possible to interrogate more areas of both testis for the presence or absence of indicative biomarkers than can be accomplished using currently offered invasive testis mapping and biopsy techniques. That is, on current clinical MR scanners using ¹H magnetic resonance spectroscopic imaging (MRSI) techniques previously used for studying prostate cancer, both testicles could be assayed for the presence of metabolomic biomarkers from contiguous 0.16 cm³ voxels (5.5 mm×5.5 mm×5.5 mm), a voxel being defined as the measure of a volume of tissue examined by MRS.

### EXAMPLES

### Example 1

### ¹H MRS of Biopsied Testis Samples

Testis biopsies from men undergoing vasectomy reversal were subject to histologic review to confirm normal spermatogenesis. Infertile men being evaluated for NOA had both histologic review of the biopsy specimen and cytologic review of the FNA mapping findings from remainder of the biopsied testis. Testis biopsies were evaluated according to the criteria of Levin, Hum. Pathol, 1979, 19:569-584. Biopsy patterns were categorized as: normal spermatogenesis, hypoplasia or hypospermatogenesis, complete or early maturation arrest (EMA), SCO (Sertoli cell-Only), incomplete or late maturation arrest (LMA) or other (including sclerosis). If a biopsy contained a single histologic pattern throughout the specimen, it was deemed a pure pattern. If biopsies exhibited two or more patterns, then a mixed pattern was assigned. Cytologic findings from FNA mapping from the remainder of the biopsied testis were classified histologically as reported by Meng et al., Human Reprod., 2000, 15:1973-1977. To reduce testis phenotypic variability as much as possible, only pure cytologic patterns from FNA mapping that corresponded to pure histologic patterns were included for SCO patients in this study.

Frozen thawed testis tissues were weighed (mean 13.64±6.89 mg) and placed into custom designed 20 or 35 μl leak-proof zirconium rotors containing 3.0 μl D₂O+0.75% TSP (3-(trimethylsilyl)-propionic-2,2,3,3-d4 acid sodium salt). ¹H-MRS data were acquired at 11.7 T, 1° C., and 2250 Hz spin rate using a Varian INOVA spectrometer equipped with a 4 mm gHX nanoprobe. Quantitative 1-D spectra were acquired with 2 s relaxation, 2 s pre-saturation, 2 s acquisition (TR=6 s), 40,000 points, 20,000 Hz spectral width and 256 transients. The ERETIC method was used as a quantitative concentration standard (Tessem et al., Magn. Reson. Med., 2008, 60:510-516). Data were quantified with a custom version of quantification software based on semi-parametric quantum estimation (QUEST), called high resolution-QUEST (HR-QUEST), adapted for analysis of short-echo time 1H-MRS spectra containing 40,000 points (Ratiney et al., NMR Biomed., 2005, 19:1-13). Basis set spectras of 19 metabolites were collected in solution and incorporated into the HR-QUEST fitting routine. The main MR-observable ¹H metabolites in the human testis—choline (Cho), creatine (Cr), glutamate (Glu), glutamine (Gln), lactate (Lac), myo-inositol (ml), phosphocratine (PCr), phosphocholine (PC), phosphoethanolamine and taurine (Tau)—were quantified and evaluated. Peaks from known macromolecules and unidentified compounds were also included as part of the basis set. HR-QUEST estimated the background signal using a Hankel-Lanczos singular value decomposition (HLSVD) algorithm and iterated between fitting the metabolites and modeling the background six times. Finally, concentrations were calculated relative to the peak area of the ERETIC signal.

Two dimensional total correlation spectroscopy (TOCSY) ¹H-MRS data were also acquired for each biopsy sample and used to further quantify the relative amounts of PC glycerol phosphocholine (GPC), phosphoethanolamine (PE) and glycerol phosphoethanolamine as these metabolites overlap in the 1-D ¹H-MRS spectra of the testes. The TOCSY spectra were acquired using a rotor synchronized adiabatic (WURST-8) mixing scheme with 1 s presaturation delay, 0.2 s acquisition time, 40 ms mixing time, 24 transients/increment, 20,000×6000 Hz spectral width, 4096×64 complex points, time approximately 1 h (Zektzer et al., Magn, Res. Med., 2005, 53:41-48).

An exploratory study using one-way ANOVA was performed to discern which metabolites, if any, would be significant indicators for the three histologies. A normality test was performed to assess for normal distribution.

Logistic regression analysis was performed using the binary dependent variable of testis biopsy histology (normal or SCO) and the independent variables, age and a solitary metabolite. For metabolites that had age-adjusted P-values <0.05, the predicted probability of ‘normal spermatogenesis’ for the EMA testis tissue samples using the logistic regression equation for normal versus SCO patients was modeled. Odds ratios (OR) with 95% confidence intervals were determined.

A total of 27 patient biopsies were evaluated from men at vasectomy reversal or from men with non-obstructive azoospermia. Patients were identified on biopsy and/or FNA mapping as having normal (n=9), MA (early, n=5 and late, n=4) or pure SCO (n=9) histology. The mean age of the normal-fertile group was older than that of either of the other groups.

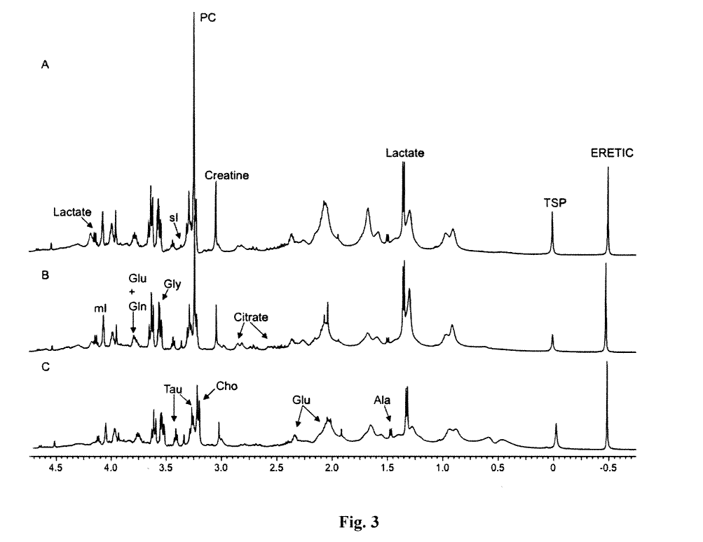

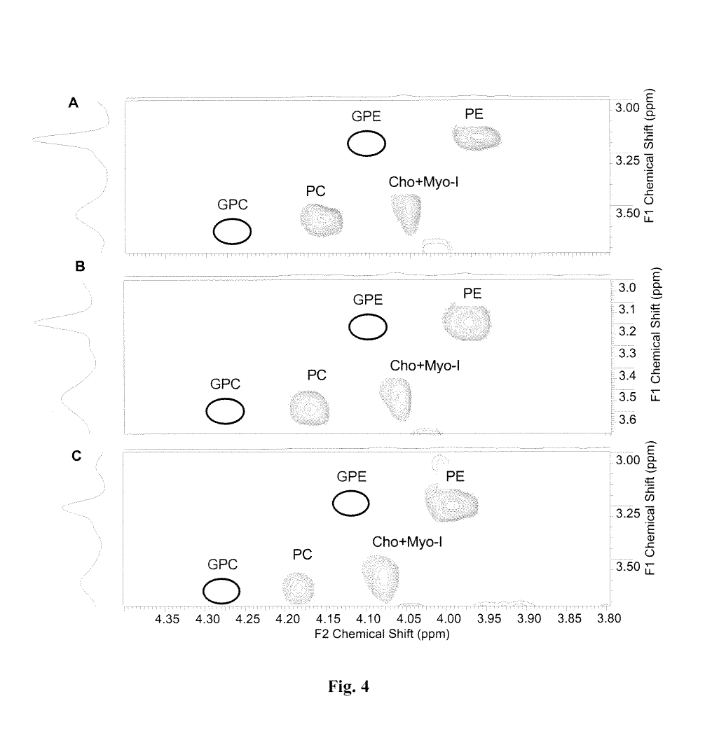

Representative 1-D ¹H-MRS spectra from biopsies taken from patients having normal (A), MA (B) and SCO histology (C) are shown in FIG. 3. Spectral peaks are labeled with their corresponding metabolites and have been scaled to the ERETIC peak so they visually reflect true and actual differences in metabolite concentration. Note that the spectral peak labeled PC is markedly elevated in normal compared with SCO patients and the PC peak for the MA patients is between that of the normal and SCO groups. The GPC and PC resonances cannot be resolved in the 1-D testes spectra as they overlap. However, the 2-D TOCSY spectra of the same testis samples indicate that the PC peak, which is composed of both GPC and PC, was predominantly composed of PC (FIG. 4). In FIG. 4, it is shown that the one-dimensional ¹H NMR is composed predominantly of PC. The circles labeled GPC indicate where glycerol phosphocholine would appear were it present. The other substances present are GPE, glycerol phosphoethanolamine, PE, phosphoethanolamine and Cho+Myo-I, choline plus myo-inositol. In a separate analysis, the mean concentrations of taurine were significantly different at 0.96, 2.46, and 1.73 mmol/Kg for the normal, SCO or MA group, respectively (OR=0.29, CI95% (0.09−0.93) p=0.037). This suggests that taurine may also have potential as a predictor of sperm in the testicle.

### Example 2

### ¹H MRS of In Vivo Testis

Infertile men were referred by their urologist for a MRI/¹H MRSI exam of their testes in order to assess the presence and spatial distribution of elevated phosphocholine, a biomarker of viable sperm in the testicle. Patients were consented and screened for any MRI contraindications. MR imaging studies were performed on a 3.0-Tesla whole body MR scanner (Signa; GE Medical Systems, Milwaukee, Wis.). Patients were scanned using the body coil for excitation and a 6″ surface coil (GE Medical Systems, Milwaukee, Wis.) for signal reception. Following a localizer sequence, T2-weighted fast spin-echo images of the testicles were acquired with the following parameters: TR/effective TE 5000/96 ms, echo train length=16, slice thickness=3 mm, interslice gap=0 mm, field of view=14 cm, matrix 256×192, anteroposterior frequency encoding, and 3 excitations.

After review of the axial T2-weighted images, a MR spectroscopic imaging volume was selected to maximize coverage of both testicles. Three-dimensional MR spectroscopic imaging data were acquired using water and lipid suppressed double-spin echo point-resolved spectroscopy sequence technique with spectral-spatial pulses for the two 180° excitation pulses, and an interleaved flyback echo-planar readout, optimized for the quantitative detection of testicular metabolites. Outer voxel saturation pulses were employed to further sharpen volume selection and to conform the selected volume to the shape of the testicles. Data sets were acquired as 16×10×8 phase-encoded spectral arrays (1280 voxels with a spatial resolution of 0.16 cm³), TR/TE 85/2000 ms, and a 5-minute acquisition time. Three-dimensional MR spectroscopic imaging data were processed off-line utilizing in-house software previously developed specifically for 3D-MR spectroscopic imaging studies. Integrated peak area values for testicular total choline (PC+GPC+choline), creatine, myoinositol, taurine, and lactate were automatically calculated for each voxel. MR spectroscopic imaging data (spectra and associated metabolic images) were overlaid on the corresponding axial T2-weighted images.

The spectra obtained in the above study provided proof of principle that in vivo ¹H MRS produces the same sort of information as that obtained from ex vivo testis biopsy samples. Thus, similar to the trends and patterns in phosphocholine (PC) concentrations discovered in the analysis of ex vivo testis biopsies, and the peak area ratios of PM/β-ATP, PM/PD and Pi/PM from ³¹P MRS in vivo scanning of men, characteristic PC concentrations or ratios of PC to other PMs, PDs or Pi's obtained from in vivo ¹H MRS can be used to define the presence of sperm, areas of ischemia, and other abnormalities that affect testis function. Such an analysis would be useful in determining: 1) if a testis contains viable sperm for pregnancies, 2) if a testis is healthy, unhealthy or ischemic and 3) if abnormalities within a testis are malignant or benign. The technology is also expected to be useful to determine the relative reproductive toxicity of various drugs on spermatogenesis or the effect of varicocele or varicocele repair on testis function.

1. A method, comprising,
obtaining an in vivo ¹H magnetic resonance spectroscopy spectrum of a testis of a subject;
comparing the subject testis spectrum with a spectrum of a testis of a control subject; and,
diagnosing the condition of the testis of the subject.

2. The method of claim 1, wherein the condition of the testis being diagnosed comprises the presence or absence of sperm and attendant fertility potential.

3. The method of claim 2, wherein diagnosing the presence of sperm comprises comparing phosphocholine, taurine and potentially other metabolic signals of the two spectra.

4. The method of claim 3, wherein the subject has undergone a surgical intervention for treatment of fertility or infertility.

5. The method of claim 4, wherein the surgical intervention is a varicocele repair, vasectomy, vasectomy reversal, tumor excision, or orchidopexy.

6. The method of claim 2, wherein the subject has undergone medical treatment for infertility.

7. The method of claim 6, wherein the medical treatment comprises administration of clomiphene citrate, tamoxifen or other selective estrogen receptor modulators, other hormonal manipulation through FSH or LH injections, correction of systemic medical disease, the use non-hormonal treatments, dietary supplements, antioxidants, vitamins and herbal remedies.

8. The method of claim 1, wherein obtaining the subject spectrum and the control subject spectrum comprises employing a spectrum-enhancing technique.

9. The method of claim 8, wherein the spectrum-enhancing technique is selected from the group consisting of water suppression, lipid suppression, stimulated echo acquisition mode (STEAM) spectroscopy, point resolved spectroscopy (PRESS), chemical shift imaging (CSI), magnetic resonance spectroscopic imaging (MRSI), short T1 inversion recovery (STIR), high resolution MRS, magic angle spinning and any combination thereof.

10. The method of claim 1, wherein the condition of the testis being diagnosed is the effect of exposure to a pharmacological agent, exposure to a toxic agent, exposure to abnormal physical conditions including excessive heat or cold, exposure to radiation therapy, exposure to chemotherapy, exposure to a disease or any combination of the foregoing on the production of sperm.

11. The method of claim 1, wherein the condition of the testis being diagnosed is the presence of testicular disease.

12. The method of claim 11, wherein the testicular disease is cancer, cryptorchidism, benign tumors such as Leydig cell, stromal or adenoid tumors, effect of varicocele, medications or exposures that include toxins, and the effects of prolonged obstruction from infection, inflammation and vasectomy.

13. The method of claim 1, wherein the subject and control subject are human beings.
